(19) United States
(12) Patent Application Publication
(10) Pub. No.: US 2006/0020166 A1
(43) Pub. Date: Jan. 26, 2006

(54) Protection of sensitive instruments

(76) Inventor: Jonathan Berall, Brooklyn, NY (US)

Correspondence Address: Jonathan Berall, 173 Columbia Hts, Brooklyn, NY, 11201, US

(21) Appl. No.: 11/185,769

(22) Filed: Jul. 21, 2005

Related U.S. Application Data
(60) Provisional application No. 60/590,140, filed on Jul. 21, 2004.

(51) Int. Cl. A61B 1/267; A61B 1/00

(52) U.S. Cl. 600/121; 600/186

(57) ABSTRACT

This patent will describe a method whereby sensitive instruments can be protected from damage and contamination. Sensitive instruments that need to be sterilized after use is a major problem for the medical field. Most camera systems fall into that category, limiting their use and denying the public the benefit of their use. The presently available protection/sterilization methods, such as autoclaving or sterilization solutions, would result in electrical damage to the camera or are totally impractical, such as nitrous oxide or radiation, for many potential camera applications.

### BACKGROUND OF INVENTION

1. Field of Invention

The invention relates to a clear/transparent protective covering to be used with video-modified medical devices.

2. Relevant Prior Art

There are many previous devices that have been used as protective covers for medical equipment. Some of the most pertinent are U.S. Pat. Nos. 5,695,454; 5,347,995 and 4,834,077. However, these Patents refer to covers to be used specifically with standard laryngoscopes and standard laryngoscope blades. The purpose of the current design is a generic covering to be modified for use with U.S. Pat. No. 5,827,178, Berall, which describes the video-laryngoscope.

And also with various video-modified medical devices, such as video-modified otoscopes and ophthalmoscopes and the Portable Handheld Medical Diagnostic Tool as per U.S. Provisional Application No. 60/590,139

### SUMMARY OF THE INVENTION

A primary object of the present invention is to provide a protective covering for a wide range of medical diagnostic tools, particularly ones containing audio/video features.

A further object is for the protective covering to provide a sterile environment by fully covering the device and connections to the handle therefore preventing contaminant buildup.

A further object is the unique design of the placement of the securing mechanism that would specifically allow full use of the video screen and output connectors to the data-receiving units.

A further object is for the protective covering to be easily removed, disposed of and replaced.

These and other objectivess will be achieved through the use of transparent polymers.

The clear protective cover will include flexible and rigid designs

### BRIEF DESCRITION OF THE DRAWINGS

Throughout the drawings the same reference characters indicate the same or similar parts.

FIG. 1 is an illustration of the clear/transparent protective cover (c/tpc) and securing element, generic form, flexible.

FIG. 2 Front View of the c/tpc attached to the handle of a video modified instrument, in this illustration also incorporating the concept of the “camcorder handle”

FIG. 3 Side View of the c/tpc attached to the handle and covering a video-modified laryngoscope.

### DESCRIPTION OF THE PREFERRED EMBODIMENT

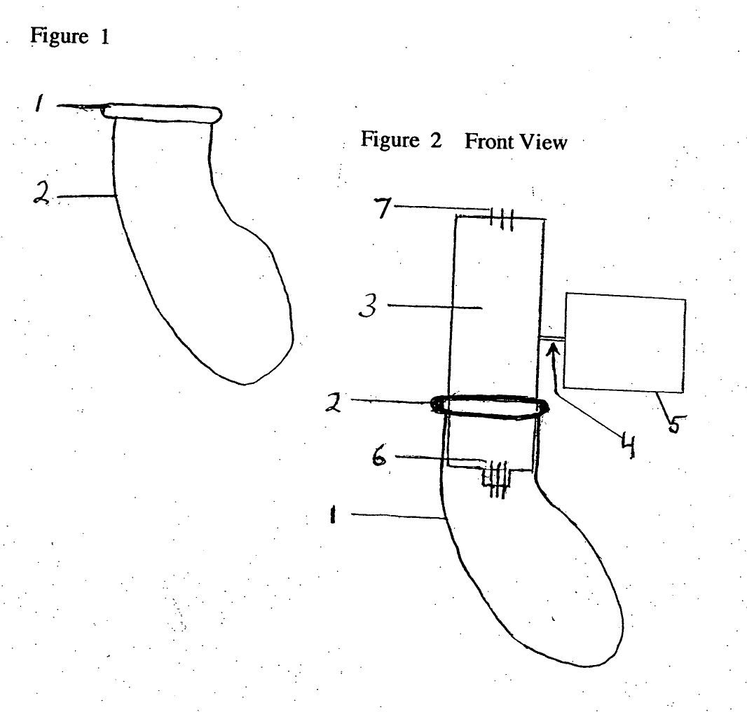

FIG. 1 illustrates the basic generic, flexible design of the clear/transparent Protective Cover for Sensitive Instruments.

It illustrates a clear/transparent protective cover (c/tpc) 2 and a securing element 1 for securing the c/tpc 2 to the instrument handle 3.

FIG. 2 illustrates securing element 2 of the c/tpc 1 to the handle 3 of a video-modified instrument FIG. 2 illustrates that the securing element 2 is placed specifically between the connectors on the handle to the video attachment 6 and the connectors for the screen 4. When the handle is equipped as a camcorder the securing element 2 will be placed between the connectors 6 and the connectors to the screen 4 and also between the connectors on the handle to the video attachment 6 and the information transfer system and the battery recharge system 7.

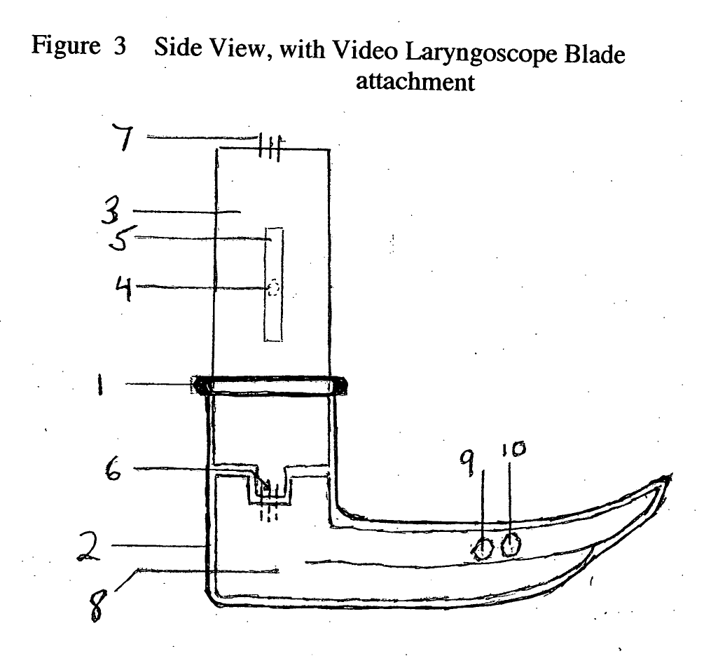

FIG. 3 illustrates FIG. 2 with the addition of the video-laryngoscope 8 to illustrate the light 9 and camera/image receiving elements 10, and their inclusion within the c/tpc 2.

I claim:

1. A clear/transparent plastic covering specifically designed for use in video-modified medical instruments, such as the video-laryngoscope.

2. As in claim 1, the clear plastic covering will permit clear image reception by the video-imaging features.

3. As in claim 1, further including a specific design of the placement of the securing mechanism to allow full use of video-modified medical instruments.

4. As in claim 1, the clear plastic cover will protect from contamination and physical damage to the video systems and the connectors.

5. As in claim 3, the design will permit utilization of the screen element.

6. As in claim 3, the design will permit connections to electrical systems/computers/monitors.

7. As in claim 3, the covering plastic (c/tpc) will entirely cover attachments, such as a video laryngoscope blade, and the connectors to the handle.

8. As in claim 3, the c/tpc will be secured to the handle of the video-modified instrument between the connections to the attachment and that of the screen.

9. As in claim 8 the c/tpc will be secured to the handle between the connection to the attachment and the information transfer and battery recharging systems

10. As in claim 1, the c/tpc will be form fitted and be flexible and/or rigid.

11. As in claim 1, securing system to handle through the use of several mechanisms, such as adhesives, latches, etc.
